(19) United States
(12) Patent Application Publication
(10) Pub. No.: US 2014/0288443 A1
(43) Pub. Date: Sep. 25, 2014

(54) MONITORING SYSTEM

(71) Applicant: MURATA MANUFACTURING CO., LTD., Nagaokakyo-shi (JP)

(72) Inventors: Ulf MERIHEINÄ, Soderkulla (FI); Pekka KOSTIAINEN, Helsinki (FI)

(73) Assignee: MURATA MANUFACTURING CO., LTD., Nagaokakyo-shi (JP)

(21) Appl. No.: 14/219,357

(22) Filed: Mar. 19, 2014

(30) Foreign Application Priority Data
Mar. 22, 2013 (FI) 20135274

(51) Int. Cl. A61B 5/021

(52) U.S. Cl. CPC A61B 5/02108
USPC 600/485

(57) ABSTRACT

A device, system and method for monitoring blood pressure information of a user. A device is configured with first and second pressure sensors, a fastening element, and a processing component. In the method the first pressure sensor is detachably attached to a first position and the second pressure sensor to a second position on the outer surface of a skin of the user. The pressure sensor generate signals that vary according to deformations of the skin in response to an arterial pressure wave expanding or contracting a blood vessel underlying the skin. The first signal and the second signal are used to compute at least one output value that represents a detected characteristic of the progressing arterial pressure wave of the user.

### FIELD OF THE INVENTION

The present invention relates to monitoring vital signs of a user and especially to a device, system, method and a computer program product for monitoring blood pressure information of a user according to preambles of the independent claims.

### BACKGROUND OF THE INVENTION

Statistics of World Health Organization report that in 2002 cardiovascular diseases represented approximately one third of all reported deaths in non-communicable diseases globally. These diseases are considered a severe and shared risk, and a majority of the burden is in low- and middle-income countries. One factor that increases the risk of heart failures or strokes, speeds up hardening of blood vessels and reduces life expectancy is Hypertension, HTN (also called as High Blood Pressure, HBP).

Hypertension is a chronic health condition in which the pressure exerted by circulating blood upon the walls of blood vessels is elevated. In order to ensure appropriate circulation of blood in blood vessels, the heart of a hypertensive person must work harder than normal, which increases the risk of heart attack, stroke and cardiac failure. However, healthy diet and exercising can significantly improve blood pressure control and decrease the risk of complications, efficient drug treatments are also available. It is therefore important to find persons with elevated blood pressures and monitor their blood pressure information on a regular basis.

During each heartbeat, blood pressure varies between a maximum (systolic) and a minimum (diastolic) pressure. A traditional non-invasive way to measure blood pressure has been to use a pressurized cuff and detect the pressure levels where the blood flow starts to pulsate (cuff pressure exceeds diastolic pressure) and where there is no flow at all (cuff pressure exceeds systolic pressure). However, it has been seen that users tend to consider the measurement situations, as well as the pressurized cuff tedious and even stressing, especially in long-term monitoring. Also the well-known white-coat syndrome tends to elevate the blood pressure during the measurement, and lead to inaccurate diagnoses.

The patent publication U.S. Pat. No. 6,533,729 discloses a blood pressure sensor that includes a source of photo-radiation, an array of photo-detectors, and a reflective surface that is placed adjacent to the location where the blood pressure data is to be acquired. Blood pressure fluctuations translate to deflections of the patient's skin and these deflections show as scattering patterns detected by the photo-detectors. The solution relieves users of cuffs and compressors, but it requires a relatively complicated calibration procedure using known blood pressure data and scattering patterns, which are obtained while the known blood pressure is obtained at a known hold down pressure. During data acquisition, scattering patterns are linearly scaled to the calibrated values of signal output and hold down pressure.

A patent application publication US2005/0228299 discloses a patch sensor for measuring blood pressure without a cuff. Also this solution requires a separate calibration process that applies a conventional blood pressure cuff to generate a calibration table to be used in subsequent measurements.

### BRIEF DESCRIPTION OF THE INVENTION

The object of the present invention is to provide an improved non-invasive blood pressure information monitoring solution where at least one of disadvantages of the prior art are eliminated or at least alleviated. The objects of the present invention are achieved with a device, system, method and a computer program product according to the characterizing portions of the independent claims.

The preferred embodiments of the invention are disclosed in the dependent claims.

The present invention is based on use of a device that includes two pressure sensors detachably attached to the arm of a user and a processing element that transforms signals from the pressure sensors to output values. The configuration is unnoticeable, simple and very easily calibrated, still it provides very accurate results.

### BRIEF DESCRIPTION OF THE FIGURES

In the following the invention will be described in greater detail, in connection with preferred embodiments, with reference to the attached drawings, in which

FIG. 1 illustrates functional elements of an embodiment of a device;

FIG. 2 illustrates functional configuration of a blood pressure information monitoring system;

FIG. 3 illustrates stages of a method for calibrating the device;

FIG. 4A illustrates a first arm position used in device calibration;

FIG. 4B illustrates a second arm position used in device calibration;

FIG. 4C illustrates a third arm position used in device calibration;

FIG. 5A illustrates a first arm position of a position-assisted calibration;

FIG. 5B illustrates a second arm position of a position-assisted calibration; and

FIG. 5C illustrates a third arm position of a position-assisted calibration.

### DETAILED DESCRIPTION OF SOME EMBODIMENTS

The following embodiments are exemplary. Although the specification may refer to “an”, “one”, or “some” embodiment(s), this does not necessarily mean that each such reference is to the same embodiment(s), or that the feature only applies to a single embodiment. Single features of different embodiments may be combined to provide further embodiments.

In the following, features of the invention will be described with a simple example of a device architecture in which various embodiments of the invention may be implemented. Only elements relevant for illustrating the embodiments are described in detail. Various implementations of blood measurement devices and blood pressure information monitoring systems comprise elements that are generally known to a person skilled in the art and may not be specifically described herein.

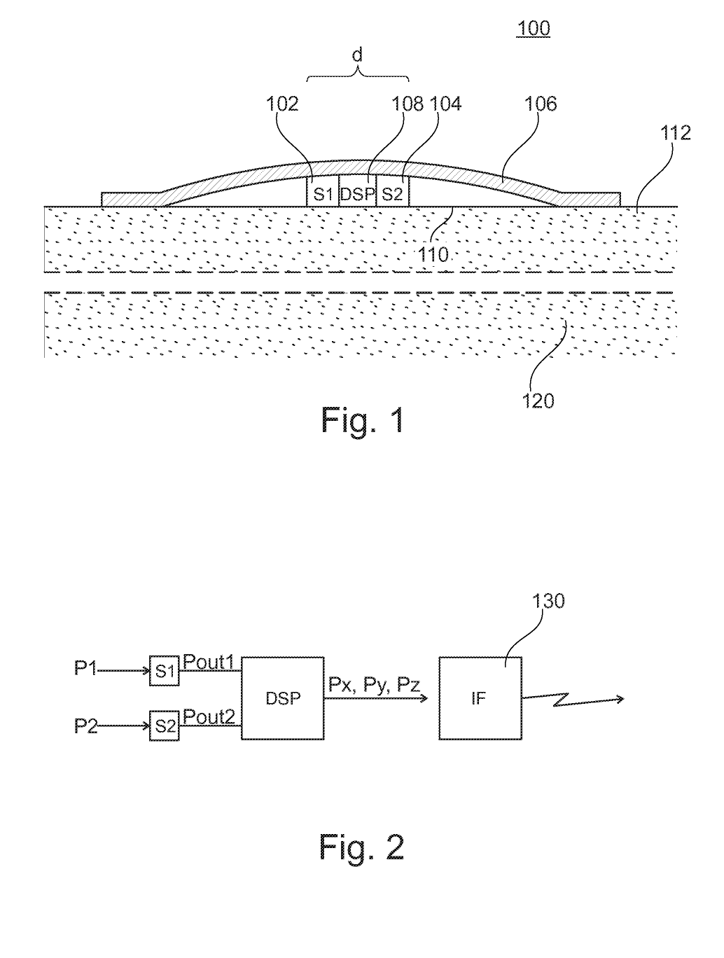

The monitoring system according to the invention comprises a device that generates one or more output values that represent detected characteristics of arterial pressure waves of a user. These values may be used as such or be further processed to indicate blood pressure information of the user. The block chart of FIG. 1 illustrates functional elements of an embodiment of a device 100 according to the present invention. It is noted that the Figure is schematic; some proportions of the elements may be exaggerated to demonstrate the functional concepts of the embodiment. The device 100 comprises a first pressure sensor 102, a second pressure sensor 104, a fastening element 106, and a processing component 108.

A pressure sensor refers here to a functional element that converts ambient pressure into mechanical displacement of a diaphragm, and translates the displacement into an electrical signal. It is noted that the device 100 comprises at least the two pressure sensors. It is clear to a person skilled in the art that additional pressure sensors may be included to the device without deviating from the scope of protection. Any two pressure sensors of the pressure sensors included in a device may be applied in the claimed manner. Advantageously capacitive high resolution pressure sensors are applied due to their low power consumption and excellent noise performance. Other types of pressure sensors, for example piezoresistive pressure sensors, may be applied, however, without deviating from the scope of protection. The first pressure sensor 102 is detachably attached to a first position, and the second pressure sensor 104 is detachably attached to a second position on the outer surface 110 of a skin 112 of a user. The first position and the second position are separated by a predefined sensor distance d. The positions are selected such that the sensors are placed along a blood vessel 120 underneath the skin of the user. The positions may be, for example, in an arm of a user. Other positions on the body of the user may be applied as well within the scope of protection.

The pressure sensors are attached to the skin with a fastening element 106 such that when an arterial pressure wave of blood expands or contracts the blood vessel 120 underlying the skin, the skin deforms and the pressure between the skin and the fastening element varies according to deformations of the skin. The fastening element 106 refers here to mechanical means that may be applied to position the pressure sensors 102, 104 into contact with the outer surface 110 of the skin 112 of the user. The fastening element 106 may be implemented, for example, with an elastic or adjustable strap. The pressure sensors 102, 104 and any electrical wiring required by their electrical connections may be attached or integrated to one surface of at least part of the strap. Other mechanisms may be applied, and fastening element 106 may apply other means of attachment, as well. For example, fastening element 106 may comprise easily removable adhesive bands to attach the pressure sensors on the skin.

The device comprises also a processing component 108 that is electrically connected to the first pressure sensor 102 and the second pressure sensor 104 to input signals generated by the pressure sensors for further processing. The processing component 108 illustrates here any configuration of processing elements included in the device 100. Advanced microelectromechanical pressure sensors are typically packaged sensor devices that include a micromachined pressure sensor and a measuring circuit. In addition, the device 100 may include a further processing element into which pre-processed signals from the pressure sensor are delivered through predefined sensor device interfaces.

A processing component is a combination of one or more computing devices for performing systematic execution of operations upon predefined data. The processing component essentially comprises one or more arithmetic logic units, a number of special registers and control circuits. The processing component may comprise or may be connected to a memory unit that provides a data medium where computer-readable data or programs, or user data can be stored. The memory unit may comprise volatile or non-volatile memory, for example EEPROM, ROM, PROM, RAM, DRAM, SRAM, firmware, programmable logic, etc.

FIG. 2 illustrates functional configuration of a blood pressure information monitoring system 200 that includes the device 100 of FIG. 1. Accordingly, the first pressure sensor 102 in the first position is exposed to pressure P1, and is configured to generate a first signal Pout1. The first signal corresponds to a pressure between the fastening element and the skin of the user, which pressure varies according to deformations of the skin when an arterial pressure wave expands or contracts a blood vessel underneath the skin in the first position. Correspondingly, the second pressure sensor 104 is exposed to pressure P2, and is configured to generate a second signal Pout2. The second signal corresponds to a pressure between the fastening element and the skin of the user, which pressure varies according to deformations of the skin in response to the arterial pressure wave expanding or contracting the blood vessel underlying the skin in the second position.

The first signal Pout1 and the second signal Pout2 are input to the processing component that is configured to use them to compute one or more output values Px, Py, Pz, each of which represents a detected characteristic of the arterial pressure wave of the user. The detected characteristic may be, for example, detected pressure exerted by the arterial pressure wave upon the walls of the underlying blood vessel, a speed of propagation of the arterial pressure wave, or shape of the waveform of the arterial pressure wave. These output values may be output to the user as such through a user interface included or integrated to the device, or they may be delivered to an external server component for further processing.

The device 100 may thus comprise, or be connected to an interface unit 130 that comprises at least one input unit for inputting data to the internal processes of the device, and at least one output unit for outputting data from the internal processes of the device.

If a line interface is applied, the interface unit 130 typically comprises plug-in units acting as a gateway for information delivered to its external connection points and for information fed to the lines connected to its external connection points. If a radio interface is applied, the interface unit 130 typically comprises a radio transceiver unit, which includes a transmitter and a receiver. The transmitter of the radio transceiver unit receives a bitstream from the processing component 108, and converts it to a radio signal for transmission by the antenna. Correspondingly, the radio signals received by the antenna are led to the receiver of the radio transceiver unit, which converts the radio signal into a bitstream that is forwarded for further processing to the processing component 108. Different radio interfaces may be implemented with one radio transceiver unit, or separate radio transceiver units may be provided for the different radio interfaces.

The interface unit 130 may also comprise a user interface with a keypad, a touch screen, a microphone, and equals for inputting data and a screen, a touch screen, a loudspeaker, and equals for outputting data.

The processing component 108 and the interface unit 130 are electrically interconnected to provide means for performing systematic execution of operations on the received and/or stored data according to predefined, essentially programmed processes. These operations comprise the procedures described for the device and the blood pressure information monitoring system.

The monitoring system may also comprise a remote node (not shown) communicatively connected to the device 100 attached to the user. The remote node may be an application server that provides blood pressure monitoring application as a service to a plurality of users. Alternatively, the remote node may be a personal computing device into which a blood pressure monitoring application has been installed.

While various aspects of the invention may be illustrated and described as block diagrams, message flow diagrams, flow charts and logic flow diagrams, or using some other pictorial representation, it is well understood that the illustrated units, blocks, apparatus, system elements, procedures and methods may be implemented in, for example, hardware, software, firmware, special purpose circuits or logic, a computing device or some combination thereof. Software routines, which are also called as program products, are articles of manufacture and can be stored in any apparatus-readable data storage medium and they include program instructions to perform particular predefined tasks. The exemplary embodiments of this invention also provide a computer program product, readable by a computer and encoding instructions for monitoring blood pressure information of a user in a device of FIG. 1 or a system of FIG. 2.

Also other characteristics of the arterial pressure wave may be measured for further blood pressure information. For example, it is easily understood that the first signal and the second signal have a similar waveform. One may select a reference point from the waveform (e.g. maximum, minimum) and detect occurrence of this reference point in the first signal and in the second signal. A time interval between an instance of the reference point in the waveform of the first signal and an instance of the reference point in the waveform of the second signal corresponds to the time needed by the pressure wave to progress from the first pressure sensor to the second pressure sensor. It is thus possible to compute a speed of propagation of the arterial pressure wave of the user by dividing the predefined sensor distance by the determined time interval. It is known that the speed of the blood pressure wave in a blood vessel may be used to indicate stiffness of the walls of the blood vessel.

As another aspect, also the shape of the waveform may be used to indicate stiffness of the walls of the blood vessel. For example, it is known that a more peaked waveform typically indicates increased stiffness in the blood vessel. It is possible to measure this estimated stiffness by computing from a waveform a value (e.g. the height of the pulse vs. the width of the pulse) and use that to indicate the interesting stiffness characteristic of the arterial pressure wave.

An important enabling factor for this novel solution has been the high resolution achieved with the advanced capacitive pressure sensors. As an example, the noise given in a data sheet of a pressure sensor component SCP1000 of Murata Electronics is 1.5 Pa@1.8 Hz and 25 μA. This corresponds to a noise density of 1.1 Pa/√Hz, which is equivalent to 0.11 mm blood assuming a density of 1 kg/l. If the predefined sensor distance is, for example, 1 cm and the gain factor is 1, a one second measurement gives a calibration error of the order of 1% (standard deviation). This is well adequate for non-invasive blood pressure measurements.

The proposed solution provides a user-friendly, stress-minimizing and still accurate method for measuring and monitoring blood pressure information. The configuration is inherently robust, because positioning of the pressure sensors in respect of the artery is not as sensitive to errors as adjusting the elements in the conventional optical arrangements. In addition, calibration of the device is quick and easy, and can be implemented without measurements with additional reference equipment.

As discussed earlier, the detected characteristic may be, for example, detected pressure exerted by the arterial pressure wave upon the walls of the underlying blood vessel. Any measurement arrangement, however, is dependent on the measurement arrangements and conditions. In order to have comparable reference values, the output values need to be calibrated. In the present configuration, calibration is simple and can be performed without additional measurement devices.

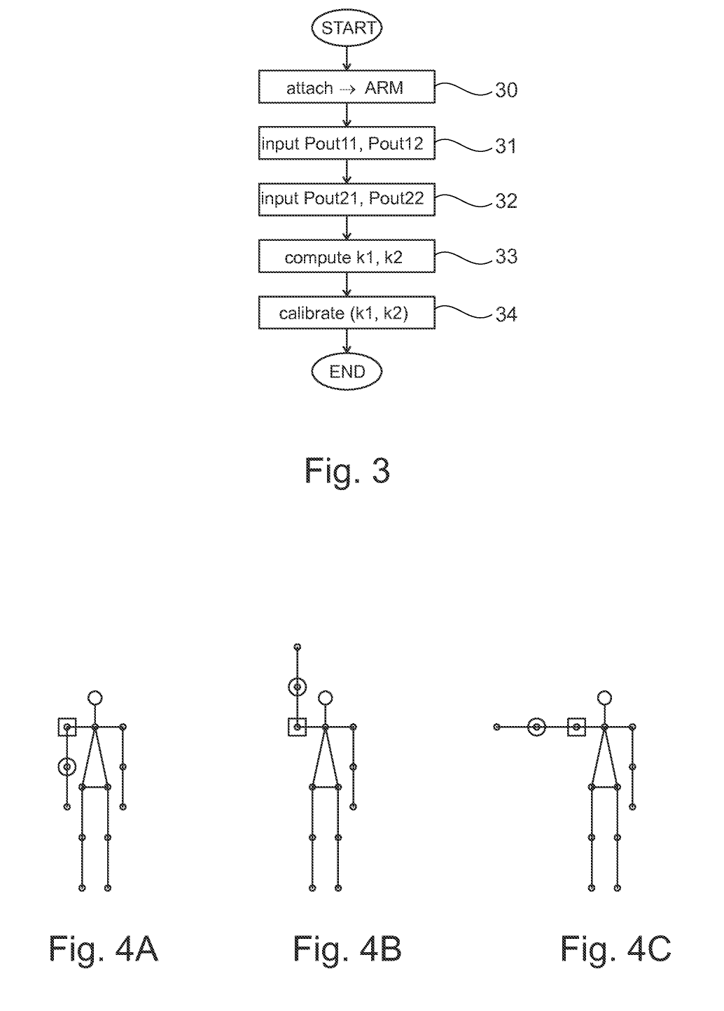

FIG. 3 illustrates stages of a method for calibrating the device of FIG. 1. The method begins by attaching (stage 30) the device on the outer surface of a skin of an arm of a user. The arm of the user is then lowered to a first arm position that is illustrated in FIG. 4A. In the first arm position the arm of the user points down such that the device is lowered to a distance h below the level of the shoulder of the user. The distance from the shoulder (denoted with a square) to the first pressure sensor is h and to the second pressure sensor (h+d). This means that:

Pout11=k1*[P−ρ*g*(h+d)]

Pout12=k2*[P−ρ*g*h]

where Pout11 stands for a reading of the first pressure sensor in the first arm position, Pout12 stands for a reading of the second pressure sensor in the first arm position, P stands for a calibrated output value representing blood pressure of the user, ρ stands for density of blood, g stands for gravity of earth and d stands for the predefined sensor distance. The first calibration readings of the first pressure sensor Pout11 and of the second pressure sensor Pout12 in a first arm position of the user are input (stage 31) to the processing component.

The arm of the user is them raised to a second arm position that is illustrated in FIG. 4B. In the second arm position the arm points up, and the device is elevated to a height h above the level of the shoulder of the user. The distance from the shoulder (denoted with a square) to the first pressure sensor is again h and to the second pressure sensor (h+d). This means that:

Pout21=k1*[P+ρ*g*(h+d)]

Pout22=k2*[P+ρ*g*h]

where Pout21 stands for a reading of the first pressure sensor in the second arm position, and Pout22 stands for a reading of the second pressure sensor in the second arm position. Other elements are denoted as discussed above. The second calibration readings of the first pressure sensor Pout21 and of the second pressure sensor Pout22 in a second arm position of the user are also input (stage 32) to the processing component.

It is now seen that there are four equations and four unknowns. It is thus possible to easily solve the functions and determine values for k1, k2, P and h. When the transfer functions k1, k2 are known (stage 33), they can be used in subsequent steps to process input values to calibrated output values (stage 34).

Calibration can be further enhanced by a further measurement in a third arm position that is illustrated in FIG. 4C. In the third arm position the arm and also the device is in the level of the shoulder. In the third arm position, the first pressure sensor and the second pressure sensor should give the same readings. In addition these readings should be the average of the readings in the first arm position and in the second arm position. If any deviations are detected, they can be easily eliminated by adjusting the transfer functions k1, k2 accordingly.

Some users may have difficulties moving their arms to exact positions, especially to the directly upright arm position at calibration. In an aspect, calibration of the device may be further enhanced by including or integrating to the device a positioning component that may be activated in at least two arm positions to indicate the height of the device, and thus of the pressure sensors at the time of calibration. The positioning may be implemented, for example, with an ultrasonic distance measurement device that is configured to measure distance from the device to an easily accessible reference point (for example roof or wall of the room where the calibration is done) and input the measured values to the processing component to be applied in the calibration equations to compute the transfer functions k1, k2. Other positioning methods may be applied, as well. For example, the device may be integrated into a smart watch or a heart rate monitoring device. Such devices may include an accurate satellite navigation system that can be also used to determine the positions in two different arm positions.

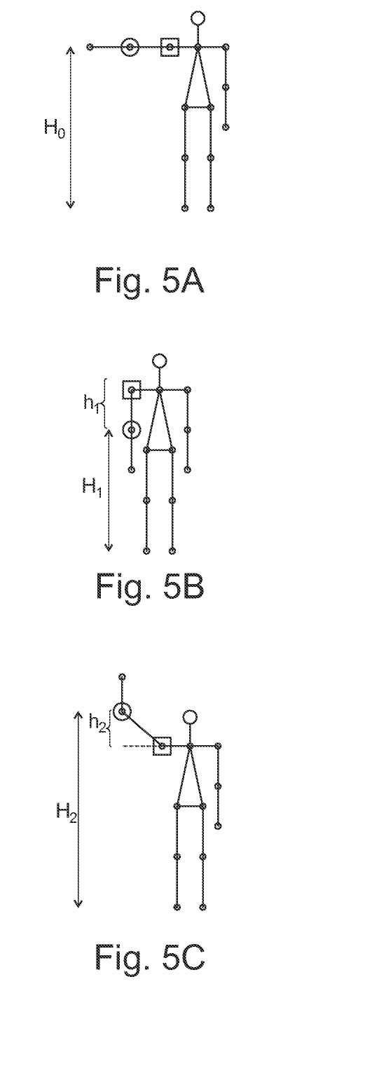

FIGS. 5A to 5C illustrate a simple example of position-assisted calibrations using the floor as a reference level. In FIG. 5A, a first measurement gives a distance HO that represents the height of the reference point. In FIG. 5B, the second measurement gives a distance H1. The distance h1 from the device to the reference level is thus h1=H0−H1. Correspondingly, in FIG. 5C, the third measurement gives a distance H2. The distance h1 from the equipment to the reference level is thus h2=H2−H0. The equations are thus:

Pout11=k1*[P−ρ*g*(h1+d)]

Pout12=k2*[P−ρ*g*h1]

Pout21=k1*[P+ρ*g*(h2+d)]

Pout22=k2*[P+ρ*g*h2]

While also h1 and h2 are known, it is simple to solve the functions and determine values for k1, k2, and P. There are more equations than unknowns, which can be further applied for improved accuracy.

It should be understood that the method of FIGS. 5A to 5C is exemplary only. Other body orientations, reference methods and positioning mechanisms may be applied without deviating from the scope of protection.

As a further aspect, the device may comprise a third pressure sensor that is exposed to ambient air pressure and is configured to generate a third signal that varies according to it. The third signal may be used, for example, to indicate the position of the arm during calibration. In these measurements, the atmospheric air pressure may be considered to increase linearly with the vertical distance to a reference point. For example, let p3₀ denote the atmospheric air pressure experienced by the device in this vertical reference point and measured with the third pressure sensor when the arm of the user points down, and p3₁ the atmospheric air pressure measured with the third pressure sensor when the arm of the user is elevated to some other arm position. The position of the arm may be estimated with equation

p3₁−p3₀=−k*Δh

where k stands for a predefined constant (e.g. ˜−8 cm/Pa) and Δh stands for the vertical distance of the device to the vertical reference point.

The third signal may also be used, for example, to facilitate computation of absolute values for the blood pressure. The blood pressure in the circulation is principally due to the pumping action of the heart, and it is measured in millimetres of mercury (mmHg), indicating positive pressure. The values computed from the signals of the first and the second pressure sensor may represent a combination of the positive pressure and the atmospheric pressure. The output value for the positive pressure within the blood vessel may be determined by subtracting the air pressure reading of the third pressure sensor from the pressure value computed with the first pressure sensor and the second pressure sensor.

It is apparent to a person skilled in the art that as technology advances, the basic idea of the invention can be implemented in various ways. The invention and its embodiments are therefore not restricted to the above examples, but they may vary within the scope of the claims

1. A device, comprising:
a first pressure sensor;
a second pressure sensor;
a fastening element for detachably attaching the first pressure sensor to a first position on the outer surface of a skin of a user, and the second pressure sensor to a second position on the outer surface of a skin of the user; wherein
the first pressure sensor is configured to generate a first signal that varies according to deformations of the skin in response to an arterial pressure wave expanding or contracting a blood vessel underlying the skin in the first position;
the second pressure sensor is configured to generate a second signal that varies according to deformations of the skin in response to the arterial pressure wave expanding or contracting the blood vessel underlying the skin in the second position;
a processing component configured to input the first signal and the second signal and compute from them at least one output value that represents a detected characteristic of the progressing arterial pressure wave of the user.

2. The device of claim 1, the detected characteristic being a detected blood pressure exerted by the arterial pressure wave upon the walls of the underlying blood vessel.

3. The device of claim 1, wherein:
the first position and the second position are separated by a predefined sensor distance;
the first signal and the second signal have a similar waveform;
the processing component is configured to identify a reference point in the waveform of the first signal and the second signal;
the processing component is configured to determine a time interval between an instance of the reference point in the waveform of the first signal and an instance of the reference point in the waveform of the second signal;
the processing component is configured to compute a speed of propagation of the arterial pressure wave of the user from the predefined sensor distance and the determined time interval.

4. The device of claim 3, wherein the processing component is configured to compute an output value representing the shape of the waveform of the first signal and the second signal.

5. The device of claim 3, wherein the processing component is configured to use the computed speed of propagation of the arterial pressure wave of the user or the output value representing the shape of the waveform of the first signal and the second signal to compute an output value that represents stiffness of walls of the underlying blood vessel.

6. The device of claim 1, wherein:
the fastening element is configured to attach the device on the outer surface of a skin of an arm of a user;
the processing component is configured to input first calibration readings of the first pressure sensor and of the second pressure sensor in a first arm position of the user, wherein in the first arm position the arm of the user points down such that the device is lowered to a distance below the level of the shoulder of the user;
the processing component is configured to input second calibration readings of the first pressure sensor and of the second pressure sensor in a second arm position of the user, wherein in the second arm position the arm of the user points up such that the device is elevated to the distance above the level of the shoulder of the user;
the processing component is configured to compute from the first calibration readings a first transfer function for the first pressure sensor and from the second calibration readings a second transfer function for the second pressure sensor;
the processing component is configured to use the first transfer function or the second transfer function to process input values to calibrated output values.

7. The device of claim 6, wherein the processing component is configured to compute the first transfer function and the second transfer function from equations:
Pout11=k1*[P−ρ*g*(h+d)]
Pout12=k2*[P−ρ*g*h]
Pout21=k1*[P+ρ*g*(h+d)]
Pout22=k2*[P+ρ*g*h]
where Pout11 stands for a reading of the first pressure sensor in the first arm position, Pout12 stands for a reading of the second pressure sensor in the first arm position, Pout21 stands for a reading of the first pressure sensor in the second arm position, Pout22 stands for a reading of the second pressure sensor in the second arm position, P stands for a calibrated output value representing blood pressure of the user, ρ stands for density of blood, g stands for gravity of earth, h stands for a distance between the device and the level of the shoulder of the user, and d stands for the predefined sensor distance.

8. The device of claim 6, wherein
the processing component is configured to input third calibration readings of the first pressure sensor and of the second pressure sensor in a third arm position of the user, wherein in the third arm position the device is in the level of a shoulder of the user; and wherein
the processing component is configured to use the third calibration readings to refine processing of input values to calibrated output values.

9. The device of claim 1, wherein the device comprises a positioning component for inputting measurement data for determining position of the device to the processing component.

10. The device of claim 9, wherein the separate positioning component is an ultrasonic distance measurement device, a satellite navigating device, or a third pressure sensor.

11. A blood pressure monitoring system, comprising a device according to claim 1.

12. A method, comprising:
monitoring blood pressure information of a user with a device, comprising a first pressure sensor, a second pressure sensor, and a fastening element;
detachably attaching the first pressure sensor to a first position on the outer surface of a skin of a user, and the second pressure sensor to a second position on the outer surface of a skin of the user;
generating with the first pressure sensor a first signal that varies according to deformations of the skin in response to an arterial pressure wave expanding or contracting a blood vessel underlying the skin in the first position;
generating with the second pressure sensor a second signal that varies according to deformations of the skin in response to the arterial pressure wave expanding or contracting the blood vessel underlying the skin in the second position; and
computing from the first signal and the second signal at least one output value that represents a detected characteristic of the progressing arterial pressure wave of the user.

13. The method of claim 12, the detected characteristic being a detected blood pressure exerted by the arterial pressure wave upon the walls of the underlying blood vessel.

14. The method of claim 12, said method further comprising:
separating the first position and the second position to a predefined sensor distance;
inputting a similar waveform for the first signal and the second signal;
identifying a reference point in the waveform of the first signal and the second signal;
determining a time interval between an instance of the reference point in the waveform of the first signal and an instance of the reference point in the waveform of the second signal;
computing a speed of propagation of the arterial pressure wave of the user from the predefined sensor distance and the determined time interval.

15. The method of claim 14, further comprising computing an output value representing the shape of the waveform of the first signal and the second signal.

16. The method of claim 14, further comprising using the computed speed of propagation of the arterial pressure wave of the user or the output value representing the shape of the waveform of the first signal and the second signal to compute an output value that represents stiffness of walls of the underlying blood vessel.

17. The method of claim 12, further comprising:
attaching the device on the outer surface of a skin of an arm of a user;
inputting first calibration readings of the first pressure sensor and of the second pressure sensor in a first arm position of the user, wherein in the first arm position the arm of the user points down such that the device is lowered to a distance below the level of the shoulder of the user;
inputting second calibration readings of the first pressure sensor and of the second pressure sensor in a second arm position of the user, wherein in the second arm position the arm of the user points up such that the device is elevated to the distance above the level of the shoulder of the user;
computing from the first calibration readings a first transfer function for the first pressure sensor and from the second calibration readings a second transfer function for the second pressure sensor; and
using the first transfer function or the second transfer function to process input values to calibrated output values.

18. The method of claim 17, further comprising computing the first transfer function and the second transfer function from equations:
Pout11=k1*[P−ρ*g*(h+d)]
Pout12=k2*[P−ρ*g*h]
Pout21=k1*[P+ρ*g*(h+d)]
Pout22=k2*[P+ρ*g*h]
where Pout11 stands for a reading of the first pressure sensor in the first arm position, Pout12 stands for a reading of the second pressure sensor in the first arm position, Pout21 stands for a reading of the first pressure sensor in the second arm position, Pout22 stands for a reading of the second pressure sensor in the second arm position, P stands for a calibrated output value representing blood pressure of the user, ρ stands for density of blood, g stands for gravity of earth, h stands for a distance between the device and the level of the shoulder of the user, and d stands for the predefined sensor distance.

19. The method of claim 17, further comprising:
inputting third calibration readings of the first pressure sensor and of the second pressure sensor in a third arm position of the user, wherein in the third arm position the device is in the level of a shoulder of the user;
using the third calibration readings to refine processing of input values to calibrated output values.

20. A computer program product embodied on a non-transitory computer-readable medium, and encoding instructions for executing a method of claim 10 in a blood pressure monitoring system.
